(19) United States
(12) Patent Application Publication
(10) Pub. No.: US 2025/0054630 A1
(43) Pub. Date: Feb. 13, 2025

(54) MACHINE LEARNING ENABLED HEPATOCELLULAR CARCINOMA MOLECULAR SUBTYPE CLASSIFICATION

(71) Applicant: Genentech, Inc., South San Francisco, CA (US)

(72) Inventors: Cleopatra KOZLOWSKI, Woodside, CA (US); Daniel RUDERMAN, San Francisco, CA (US)

(21) Appl. No.: 18/929,474

(22) Filed: Oct. 28, 2024

Related U.S. Application Data
(63) Continuation of application No. PCT/US2023/020055, filed on Apr. 26, 2023.
(60) Provisional application No. 63/337,006, filed on Apr. 29, 2022.

(51) Int. Cl. G16H 50/20; G16B 15/00; G16H 10/60

(52) U.S. Cl. CPC G16H 50/20; G16B 15/00; G16H 10/60

(57) ABSTRACT

A method for image-based hepatocellular carcinoma (HCC) molecular subtype classification may include determining, within an image depicting a plurality of cells of a biological sample, a plurality of tiles with each tile depicting a portion of the plurality of cells comprising the sample. A machine learning model may be applied to determine a molecular subtype for the portion of the plurality of cells depicted in each tile. Moreover, an overall molecular subtype for the plurality of cells depicted in the image of the biological sample may be determined based on the molecular subtype of the portion of the plurality of cells depicted in each tile of the plurality of tiles. For example, another machine learning model may be applied to determine the overall molecular subtype of the plurality of cells depicted in the image of the biological sample. Related systems and computer program products are also provided.

### CROSS-REFERENCE TO RELATED APPLICATION(S)

This application is a continuation of International Application No. PCT/US2023/020055, filed on Apr. 26, 2023, which claims priority to and the benefit of U.S. Provisional Application No. 63/337,006 filed Apr. 29, 2022, the entire content of which is hereby incorporated by reference for all purposes.

### TECHNICAL FIELD

The subject matter described herein relates generally to digital pathology and more specifically to machine learning based techniques for hepatocellular carcinoma (HCC) molecular subtype classification.

### INTRODUCTION

Hepatocellular carcinoma (HCC) is a common disease with a high mortality rate but few effective treatment options. Although combination immunotherapies, such as atezolizumab (anti-PD-L1) and bevacizumab (anti-VEGF), has demonstrated strong antitumor activity in clinical trials, a large proportion of patients still had progressive disease. In fact, a precise understanding of hepatocellular carcinoma tumor heterogeneity and the corresponding immune response mechanisms remains elusive. As such, biological insights into hepatocellular carcinoma heterogeneity remains crucial for identifying effective new therapeutic targets.

### SUMMARY

Systems, methods, and articles of manufacture, including computer program products, are provided for image-based hepatocellular carcinoma (HCC) subtype classification. In some example embodiments, there is provided a system that includes at least one processor and at least one memory. The at least one memory may include program code that provides operations when executed by the at least one processor. The operations may include: determining, within an image of a biological sample, a plurality of tiles, each tile of the plurality of tiles depicting a portion of the biological sample: applying a first machine learning model to determine a molecular subtype for the portion of the biological sample depicted in each tile of the plurality of tiles; and determining, based at least on the molecular subtype of each tile of the plurality of tiles, an overall molecular subtype for the biological sample.

In some variations, one or more features disclosed herein including the following features can optionally be included in any feasible combination. The overall molecular subtype of the biological sample may be determined by applying a second machine learning model.

In some variations, the second machine learning model may be trained to determine the overall molecular subtype by at least determining a representational encoding of the plurality of tiles.

In some variations, the second machine learning model may be further trained to assign, to a first tile of the plurality of tiles, a higher attention score than a second tile of the plurality of tiles while determining the representational encoding of the plurality of tiles.

In some variations, the higher attention score may indicate that a first molecular subtype of the first tile contributes more to the representational encoding of the plurality of tiles than a second molecular subtype of the second tile.

In some variations, the higher attention score may indicate that a first molecular subtype of the first tile is more relevant to the overall molecular subtype of the biological sample than a second molecular subtype of the second tile.

In some variations, the second machine learning model may include a multiple instance learning (MIL) model.

In some variations, the second machine learning model may include an attention mechanism.

In some variations, the operations may further include: generating a first visual representation of a reduced dimension representation of the plurality of tiles.

In some variations, the first visual representation may include one or more visual indicators configured to provide a visual differentiation between tiles of different subtypes.

In some variations, the first visual representation may be generated by at least applying, to a pixel-wise representation of each tile of the plurality of tiles, a dimensionality reduction technique.

In some variations, the dimensionality reduction technique may include one or more of a principal component analysis (PCA), a uniform manifold approximation and projection (UMAP), and a T-distributed Stochastic Neighbor Embedding (t-SNE).

In some variations, the first visual representation may be further generated to include one or more visual indications configured to provide a visual differentiation between one or more clusters of similar tiles within the plurality of tiles.

In some variations, the operations may further include: generating a second visual representation depicting the plurality of tiles organized in accordance with the one or more clusters of similar tiles.

In some variations, the operations may further include: generating a second visual representation depicting a spatial distribution of the one or more clusters of similar tiles within the biological sample.

In some variations, the one or more clusters of similar tiles may be identified by applying a cluster analysis technique.

In some variations, the cluster analysis technique may include one or more of a k-means clustering, a mean-shift clustering, a density-based spatial clustering of applications with noise (DBSCAN), an expectation-maximization (EM) clustering using Gaussian mixture models (GMM), and an agglomerative hierarchical clustering.

In some variations, the overall molecular subtype of the biological sample may be determined based at least on a quantity of each molecular subtype present within the plurality of cells.

In some variations, the operations may further include: generating, based at least on the molecular subtype of each tile of the plurality of tiles, a visual representation depicting a spatial distribution of one or more molecular subtypes within the biological sample.

In some variations, the operations may further include: generating a visual representation depicting a first tile of the plurality of tiles having a first subtype along with a second tile of the first subtype from a same biological sample or a different biological sample.

In some variations, the visual representation is further generated to depict a third tile of the plurality of tiles having a second subtype along with a fourth tile of the second subtype from the same biological sample or the different biological sample.

In some variations, the plurality of tiles may exclude one or more tiles in the image with an above-threshold proportion of a background of the image or a below-threshold mean color channel variance.

In some variations, the first machine learning model may be trained to determine the molecular subtype associated with each tile of the plurality of tiles based on a morphological pattern present within the portion of the biological sample depicted in each tile.

In some variations, the first machine learning model may include an artificial neural network (ANN).

In some variations, the biological sample may include a hepatocellular carcinoma (HCC) tissue sample. Each tile of the plurality of tiles may be assigned a molecular subtype comprising one of a cholangio-like subtype, a hepatocyte-like subtype, or a progenitor-like subtype. The overall molecular subtype of the plurality of cells depicted in the image of the biological sample may include one of the cholangio-like subtype, the hepatocyte-like subtype, or the progenitor-like subtype.

In some variations, the operations may further include: identifying, based at least on transcriptome data associated with a plurality of tumor tissue samples, a plurality of molecular subtypes.

In some variations, the plurality of tumor tissue samples may include a plurality of hepatocellular carcinoma (HCC) tumor tissue samples. The plurality of molecular subtypes may include a cholangio-like subtype, a hepatocyte-like subtype, and a progenitor-like subtype.

In some variations, the first machine learning model may be trained to assign, to each tile of the plurality of tiles, a label corresponding to one of the plurality of molecular subtypes identified based on the transcriptome data.

In some variations, the overall molecular subtype of the plurality of cells depicted in the image of the biological sample may include one of the plurality of molecular subtypes identified based on the transcriptome data.

In some variations, wherein the image may depict a plurality of cells comprising the biological sample. Each tile of the plurality of tiles may depict a portion of the plurality of cells comprising the biological sample.

In another aspect, there is provided a method for image-based hepatocellular carcinoma (HCC) subtype classification. The method may include: determining, within an image of a biological sample, a plurality of tiles, each tile of the plurality of tiles depicting a portion of the biological sample: applying a first machine learning model to determine a molecular subtype for the portion of the biological sample depicted in each tile of the plurality of tiles; and determining, based at least on the molecular subtype of each tile of the plurality of tiles, an overall molecular subtype for the biological sample.

In some variations, one or more features disclosed herein including the following features can optionally be included in any feasible combination. The overall molecular subtype of the biological sample may be determined by applying a second machine learning model.

In some variations, the second machine learning model may be trained to determine the overall molecular subtype by at least determining a representational encoding of the plurality of tiles.

In some variations, the second machine learning model may be further trained to assign, to a first tile of the plurality of tiles, a higher attention score than a second tile of the plurality of tiles while determining the representational encoding of the plurality of tiles.

In some variations, the higher attention score may indicate that a first molecular subtype of the first tile contributes more to the representational encoding of the plurality of tiles than a second molecular subtype of the second tile.

In some variations, the higher attention score may indicate that a first molecular subtype of the first tile is more relevant to the overall molecular subtype of the biological sample than a second molecular subtype of the second tile.

In some variations, the second machine learning model may include a multiple instance learning (MIL) model.

In some variations, the second machine learning model may include an attention mechanism.

In some variations, the method may further include: generating a first visual representation of a reduced dimension representation of the plurality of tiles.

In some variations, the first visual representation may include one or more visual indicators configured to provide a visual differentiation between tiles of different subtypes.

In some variations, the first visual representation may be generated by at least applying, to a pixel-wise representation of each tile of the plurality of tiles, a dimensionality reduction technique.

In some variations, the dimensionality reduction technique may include one or more of a principal component analysis (PCA), a uniform manifold approximation and projection (UMAP), and a T-distributed Stochastic Neighbor Embedding (t-SNE).

In some variations, the first visual representation may be further generated to include one or more visual indications configured to provide a visual differentiation between one or more clusters of similar tiles within the plurality of tiles.

In some variations, the method may further include: generating a second visual representation depicting the plurality of tiles organized in accordance with the one or more clusters of similar tiles.

In some variations, the method may further include: generating a second visual representation depicting a spatial distribution of the one or more clusters of similar tiles within the biological sample.

In some variations, the one or more clusters of similar tiles may be identified by applying a cluster analysis technique.

In some variations, the cluster analysis technique may include one or more of a k-means clustering, a mean-shift clustering, a density-based spatial clustering of applications with noise (DBSCAN), an expectation-maximization (EM) clustering using Gaussian mixture models (GMM), and an agglomerative hierarchical clustering.

In some variations, the overall molecular subtype of the biological sample may be determined based at least on a quantity of each molecular subtype present within the plurality of cells.

In some variations, the method may further include: generating, based at least on the molecular subtype of each tile of the plurality of tiles, a visual representation depicting a spatial distribution of one or more molecular subtypes within the biological sample.

In some variations, the method may further include: generating a visual representation depicting a first tile of the plurality of tiles having a first subtype along with a second tile of the first subtype from a same biological sample or a different biological sample.

In some variations, the visual representation is further generated to depict a third tile of the plurality of tiles having a second subtype along with a fourth tile of the second subtype from the same biological sample or the different biological sample.

In some variations, the plurality of tiles may exclude one or more tiles in the image with an above-threshold proportion of a background of the image or a below-threshold mean color channel variance.

In some variations, the first machine learning model may be trained to determine the molecular subtype associated with each tile of the plurality of tiles based on a morphological pattern present within the portion of the biological sample depicted in each tile.

In some variations, the first machine learning model may include an artificial neural network (ANN).

In some variations, the biological sample may include a hepatocellular carcinoma (HCC) tissue sample. Each tile of the plurality of tiles may be assigned a molecular subtype comprising one of a cholangio-like subtype, a hepatocyte-like subtype, or a progenitor-like subtype. The overall molecular subtype of the plurality of cells depicted in the image of the biological sample may include one of the cholangio-like subtype, the hepatocyte-like subtype, or the progenitor-like subtype.

In some variations, the method may further include: identifying, based at least on transcriptome data associated with a plurality of tumor tissue samples, a plurality of molecular subtypes.

In some variations, the plurality of tumor tissue samples may include a plurality of hepatocellular carcinoma (HCC) tumor tissue samples. The plurality of molecular subtypes may include a cholangio-like subtype, a hepatocyte-like subtype, and a progenitor-like subtype.

In some variations, the first machine learning model may be trained to assign, to each tile of the plurality of tiles, a label corresponding to one of the plurality of molecular subtypes identified based on the transcriptome data.

In some variations, the overall molecular subtype of the plurality of cells depicted in the image of the biological sample may include one of the plurality of molecular subtypes identified based on the transcriptome data.

In some variations, wherein the image may depict a plurality of cells comprising the biological sample. Each tile of the plurality of tiles may depict a portion of the plurality of cells comprising the biological sample.

In another aspect, there is provided a computer program product including a non-transitory computer readable medium storing instructions. The instructions may cause operations may executed by at least one data processor. The operations may include: determining, within an image of a biological sample, a plurality of tiles, each tile of the plurality of tiles depicting a portion of the biological sample: applying a first machine learning model to determine a molecular subtype for the portion of the biological sample depicted in each tile of the plurality of tiles; and determining, based at least on the molecular subtype of each tile of the plurality of tiles, an overall molecular subtype for the biological sample.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to hepatocellular carcinoma (HCC), it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a digital pathology system, in accordance with some example embodiments;

FIG. 2 depicts the linkage between hepatocellular carcinoma (HCC) molecular subtypes and liver epithelial cell lineage, in accordance with some example embodiments:

FIG. 3A depicts a schematic diagram illustrating an example of an image being divided into individual tiles, in accordance with some example embodiments:

FIG. 3B depicts examples of individual tiles from an image of a tumor sample, in accordance with some example embodiments:

FIG. 4 depicts a schematic diagram illustrating an example of a machine learning model trained to perform image-based molecular subtype classification, in accordance with some example embodiments:

FIG. 5A depicts a screenshot illustrating an example of a visual representation, in accordance with some example embodiments;

FIG. 5B depicts a screenshot illustrating another example of a visual representation, in accordance with some example embodiments:

FIG. 6A depicts a screenshot illustrating another example of a visual representation, in accordance with some example embodiments;

FIG. 6B depicts a screenshot illustrating another example of a visual representation, in accordance with some example embodiments:

FIG. 7 depicts a screenshot illustrating another example of a visual representation, in accordance with some example embodiments:

FIG. 8A depicts a screenshot illustrating another example of a visual representation, in accordance with some example embodiments:

FIG. 8B depicts a screenshot illustrating another example of a visual representation, in accordance with some example embodiments:

FIG. 8C depicts a screenshot illustrating another example of a visual representation, in accordance with some example embodiments:

FIG. 8D depicts a screenshot illustrating another example of a visual representation, in accordance with some example embodiments:

FIG. 8E depicts a screenshot illustrating another example of a visual representation, in accordance with some example embodiments:

FIG. 9 depicts a flowchart illustrating an example of a process for image based hepatocellular carcinoma (HCC) molecular subtype classification, in accordance with some example embodiments;

FIG. 10 depicts a graph illustrating an receiver operating characteristic (ROC) curve representative of the performance of a machine learning model trained to perform image-based molecular subtype classification, in accordance with some example embodiments:

FIG. 11 depicts a block diagram illustrating an example of a computing system, in accordance with some example embodiments.

When practical, similar reference numbers denote similar structures, features, or elements.

### DETAILED DESCRIPTION

Hepatocellular carcinoma (HCC) is a highly heterogeneous disease with complex etiological factors as well as diverse molecular and cellular dysfunctions. As noted, biological insights into hepatocellular carcinoma heterogeneity remains crucial for identifying effective new therapeutic targets. For example, in hepatocellular carcinoma (HCC), as well as other cancers, the molecular subtypes present in a tumor may serve as a crucial biomarker for predicting patient response to therapy and survival. Nevertheless, due to prohibitive costs and the scarcity of patient tumor-specific transcriptome data, conventional transcriptome based molecular subtype classification (e.g., RNA sequence based molecular subtyping) has limited practicability. Meanwhile, conventional digital pathology approaches to image-based molecular subtype classification are associated with significant outcome variability. As such, in some example embodiments, a digital pathology platform may configured to perform machine learning enabled image-based molecular subtype classification in which the molecular subtype of a tumor sample, such as a hepatocellular carcinoma (HCC) tumor sample, is determined by applying one or more machine learning models to images of the tumor sample instead of and/or in addition to transcriptome data. Thus, even in the absence of transcriptome data, the one or more molecular subtypes that are present within the tumor sample may be determined based on morphological patterns detected within the images of the tumor sample.

In some example embodiments, an image depicting the tumor sample may exhibit an overall molecular subtype that is determined based on the molecular subtype of one or more individual portions of the image. For example, the digital pathology platform may partition, into multiple tiles, an image depicting the cells of a tumor sample (e.g., a whole slide microscopic image and/or the like). Accordingly, each of the resulting tiles may depict a portion of the tumor sample. Moreover, the digital pathology platform may apply, to each tile, a first machine learning model, such as an artificial neural network (ANN), in order to determine a molecular subtype for the portion of the tumor sample depicted therein. The overall molecular subtype of the tumor sample may be determined based at least on the molecular subtype of each tile.

In some example embodiments, the overall molecular subtype of the tumor sample depicted in the image may be determined based on a quantity, such as a relative proportion, of each molecular subtype present within the tumor sample. Alternatively and/or additionally, the digital pathology platform may apply a second machine learning model to determine, based at least on the molecular subtype of each tile, the overall molecular subtype of the tumor sample. For example, the second machine learning model may be a multiple instance learning (MIL) model trained to determine the overall molecular subtype by determining a representational encoding of the tiles included in the image. In some cases, the second machine learning model may include an attention mechanism configured to assign, to each tile, an attention score representative of how relevant the molecular subtype of each tile is to the overall molecular subtype of the image of the tumor sample. Accordingly, a first tile having a first molecular subtype may be assigned a higher attention score than a second tile having a second molecular subtype if the first molecular subtype of the first tile is more relevant to the overall molecular subtype of the image than the second molecular subtype of the second tile.

In some example embodiments, the digital pathology platform may generate one or more visual representations of at least a portion of the results of the image-based molecular subtype classification performed on the tumor sample. For example, in some cases, the digital pathology platform may generate a visual representation depicting a spatial distribution of the different subtypes present within the tumor sample. Alternatively and/or additionally, the digital pathology platform may generate a visual representation in which tiles of a same molecular subtype are aligned adjacent to other tiles of the same molecular subtype from the same tumor sample and/or different tumor samples.

In some example embodiments, the digital pathology platform may generate a visual representation depicting one or more subpopulations of similar tiles present within the image of the tumor sample. In some cases, one or more subpopulations of similar tiles may be identified by applying, to a pixel-wise representation of each tile, a cluster analysis technique such as a k-means clustering, a mean-shift clustering, a density-based spatial clustering of applications with noise (DBSCAN), an expectation-maximization (EM) clustering using Gaussian mixture models (GMM), an agglomerative hierarchical clustering, and/or the like. Alternatively and/or additionally, one or more subpopulations of similar tiles may be identified by applying a dimensionality reduction technique such as a principal component analysis (PCA), a uniform manifold approximation and projection (UMAP), a T-distributed Stochastic Neighbor Embedding (t-SNE), and/or the like. The resulting reduced dimension representation of the tiles may correspond to a projection of an m-dimensional pixel-wise representation of each tile onto a lower n-dimensional subspace (where n<<m).

Accordingly, the digital pathology platform may generate a visual representation in which the distribution of the tiles provides a visual indication of similar and dissimilar tiles present within the image. In some cases, the visual representation of the reduced dimension representation of the tiles may include visual indicators (e.g., symbols of different colors, shapes, sizes, and/or the like) to enable further visual differentiation between tiles of different molecular phenotypes. In doing so, the visual representation of the reduced dimension representation of the tiles may provide a visual indication of the overlap between the different molecular subtypes present within the image of the tumor sample. Alternatively and/or additionally, the visual representation may depict a spatial distribution of similar tiles within the tumor sample. Such a visual representation may include, within the image of the tumor sample, visual indicators (e.g., symbols of different colors, shapes, sizes, and/or the like) that provide a visual differentiation between tiles from different clusters of similar tiles.

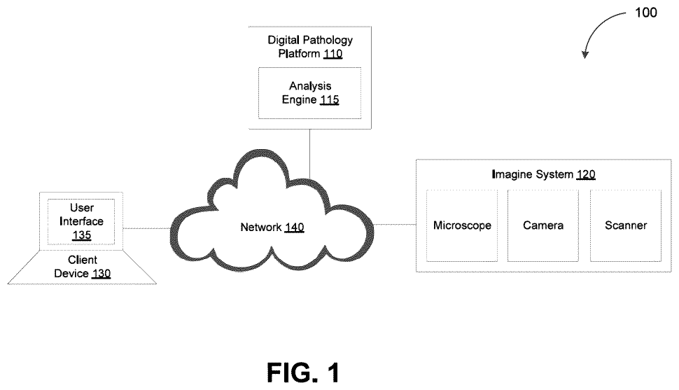

FIG. 1 depicts a system diagram illustrating an example of a digital pathology system 100, in accordance with some example embodiments. Referring to FIG. 1, the digital pathology system 100 may include a digital pathology platform 110, an imaging system 120, and a client device 130. As shown in FIG. 1, the digital pathology platform 110, the imaging system 120, and the client device 130 may be communicatively coupled via a network 140. The network 140 may be a wired network and/or a wireless network including, for example, a local area network (LAN), a virtual local area network (VLAN), a wide area network (WAN), a public land mobile network (PLMN), the Internet, and/or the like. The imaging system 120 may include one or more imaging devices including, for example, a microscope, a digital camera, a whole slide scanner, a robotic microscope, and/or the like. The client device 130 may be a processor-based device including, for example, a workstation, a desktop computer, a laptop computer, a smartphone, a tablet computer, a wearable apparatus, and/or the like.

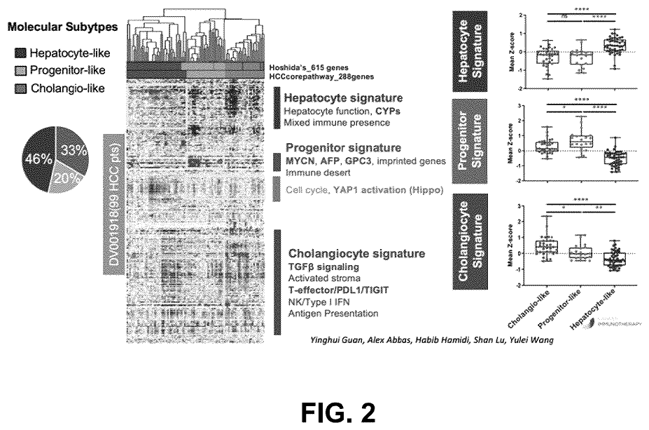

Referring again to FIG. 1, the digital pathology platform 110 may include an analysis engine 115 configured to determine, based at least on one or more images of a tumor sample, one or more molecular subtypes associated with the tumor sample. The one or more images of the tumor sample may be whole slide images (WSI) received, for example, from the imaging system 120. In some cases, the tumor sample may be a hepatocellular carcinoma (HCC) tumor sample. As shown in FIG. 2, hepatocellular carcinoma (HCC) is associated with the cholangio-like subtype, the progenitor-like subtype, and the hepatocyte-like subtype, which are identified based on the transcriptome data of various hepatocellular carcinoma tumor samples (e.g., a non-negative matrix factorization (NFM) or other cluster analysis of the transcriptome data). Each molecular subtype of hepatocellular carcinoma may be associated with a different liver epithelial cell lineage. For example, the cholangio-like subtype, the progenitor-like subtype, and the hepatocyte-like subtype are liked to cholangiocytes, bi-potent progenitors, and hepatocytes, respectively. Moreover, each molecular subtype of hepatocellular carcinoma may present a unique combination of tumor cell-intrinsic features and tumor microenvironment (TME) features. Accordingly, in hepatocellular carcinoma (HCC), as well as other cancers, the molecular subtypes present in a tumor may serve as a crucial biomarker for predicting patient response to therapy and survival.

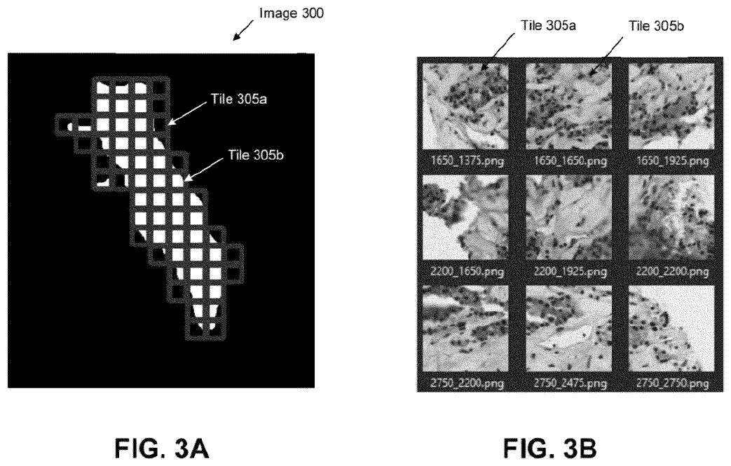

Accordingly, the analysis engine 115 may apply one or more machine learning models to determine, based at least on an image of a tumor sample, one or more molecular subtypes associated with the tumor sample. In some example embodiments, an overall molecular subtype for the tumor sample depicted in the image may be determined based on the molecular subtypes of the individual tiles within the image. For example, as shown in FIGS. 3A-B, the analysis engine 115 may determine, within an image 300 of a tumor sample, one or more tiles 305 including, for example, a first tile 305a, a second tile 305b, and/or the like. Each tile 305 may include a portion of the tumor sample depicted in the image. For example, the image 300 of the tumor sample may depict the cells present in the tumor sample, in which case each tile 305 may include a portion of the cells present in the tumor sample. Moreover, in some cases, the analysis engine 115 may exclude, from further image-based molecular subtype classification, tiles that do not depicts an above threshold quantity of the cells present in the tumor sample. Examples of tiles excluded from further image-based molecular subtype classification may include tiles with an above-threshold proportion of a background of the image, tiles with a below-threshold mean color channel variance (e.g., gray colored tiles), and/or the like.

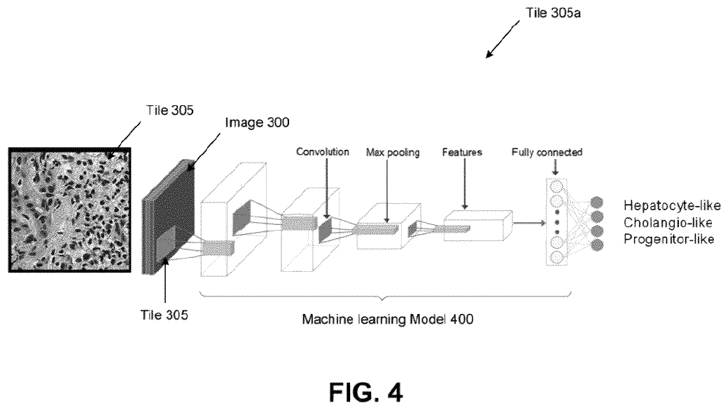

Referring now to FIG. 4, in some example embodiments, the analysis engine 115 may apply, to each tile 305 within the image 300 of the tumor sample, a machine learning model 400 trained to determine a molecular subtype for each tile 305. In the example shown in FIG. 4, the machine learning model 400 is an artificial neural network (ANN) having one or more of a convolution layer, a max pooling layer, and a fully connected layer. The machine learning model 400 may be trained based on training data including images of tumor samples (or image tiles depicting portions of a tumor sample) that have been annotated with ground-truth molecular subtype labels. As shown in FIG. 4, the fully connected layer of the machine learning model 400 may generate, based at least on the morphological features of the tile 305 extracted by the convolution layer and the max pooling layer, an output indicating the molecular subtype that is present within the tile 305.

In some example embodiments, the overall molecular subtype for the tumor sample depicted in the image 300 may be determined based on the molecular subtypes of the individual tiles 305 within the image 300. For example, in some cases, the analysis engine 115 may determine, based on a quantity of each molecular subtype present within the tumor sample, the overall subtype for the tumor sample. Alternatively and/or additionally, the analysis engine 115 may apply a second machine learning model to determine, based at least on the molecular subtype of each tile 305, the overall molecular subtype of the tumor sample depicted in the image 300. For instance, the second machine learning model may be a multiple instance learning (MIL) model trained to determine the overall molecular subtype by determining a representational encoding of the tiles 305 included in the image 300. In some cases, the second machine learning model may include an attention mechanism configured to assign, to each tile 305, an attention score representative of how relevant the molecular subtype of each tile 305 is to the overall molecular subtype of the image 300 of the tumor sample. Accordingly, the first tile 305a may be assigned a higher attention score than the second tile 305b if the first molecular subtype of the first tile 305 is more relevant to the overall molecular subtype of the image 300 than the second molecular subtype of the second tile 305b.

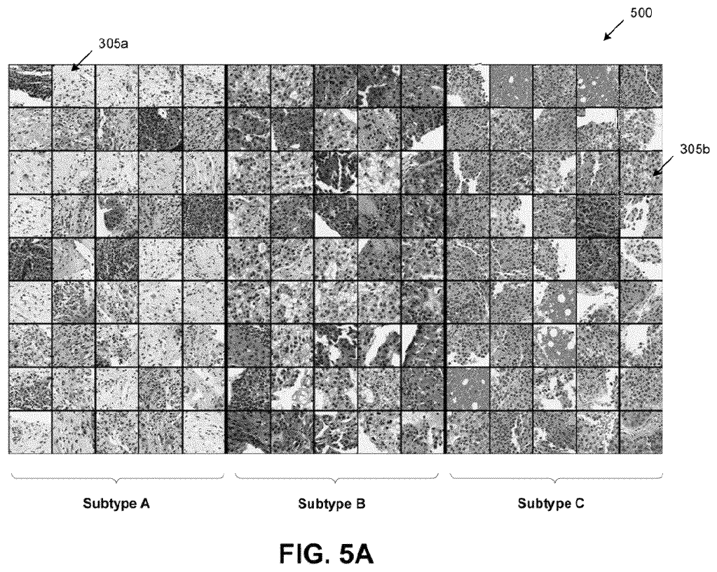

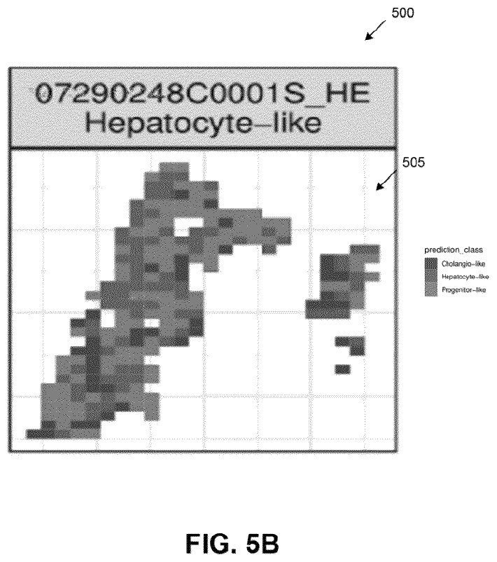

In some example embodiments, the analysis engine 115 may generate, for display in a user interface 135 at the client device 130, for example, one or more visual representations of at least a portion of the results of the image-based molecular subtype classification performed on the tumor sample. In one example shown in FIG. 5A, the analysis engine 115 may generate a visual representation 500 in which the tiles 305 from the image 300 are arranged in accordance with the molecular subtype exhibited by each of the tiles 305. For instance, as shown in FIG. 5A, the visual representation 500 may include a first grouping of tiles exhibiting a first subtype A (e.g., the cholangio-like subtype), a second grouping of tiles exhibiting a second subtype B (e.g., the hepatocyte-like subtype), and a third grouping of tiles exhibiting a third subtype C (e.g., the progenitor-like subtype).

FIG. 5B depicts another example of a visual representation 550, which may be a distribution map 505 depicting the spatial distribution of tiles of different molecular subtypes within the tumor sample shown in the image 300. In the example of the visual representation 550 shown in FIG. 5B, each tile 305 within the image 300 may be represented using a visual indicator corresponding to the molecular subtype of the tile. Accordingly, symbols of different shapes, sizes, and/or colors may be used to enable a visual differentiation between, for example, tiles of the cholangio-like subtype, the progenitor-like subtype, and the hepatocyte-like subtype.

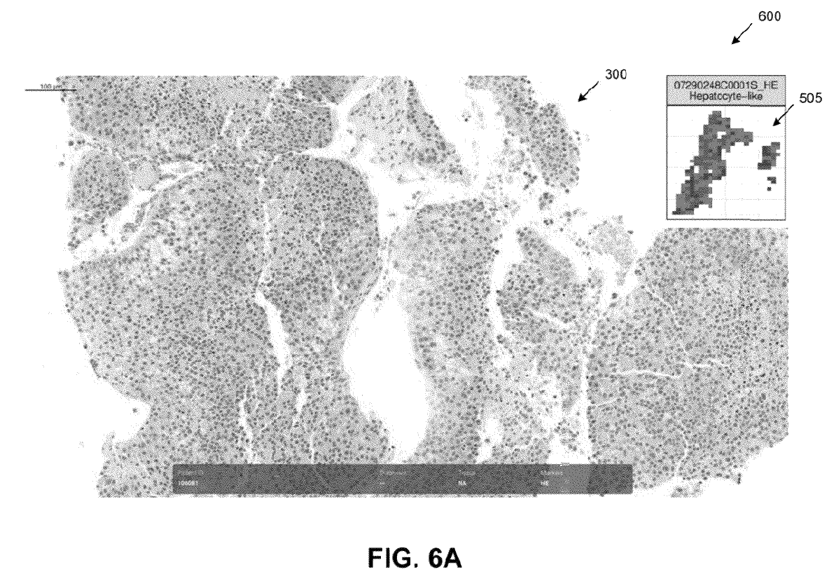

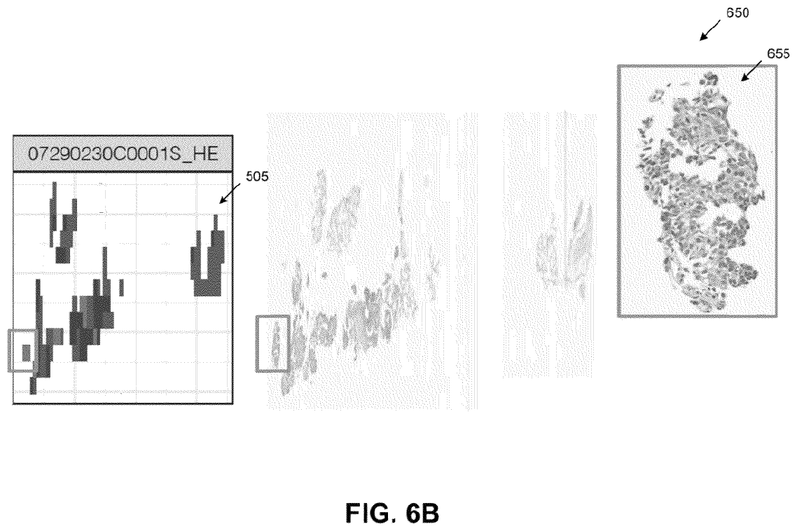

FIGS. 6A-B depicts examples of visual representations in which the distribution map 505 of tiles of different molecular subtypes within the tumor sample shown in the image 300 are juxtaposed next to the image 300 and/or a portion of the image 300. For example, FIG. 6A depicts one example of a visual representation 600 in which the distribution map 500 of tiles of different molecular subtypes within the tumor sample shown in the image 300 is superimposed over a portion of the image 300. Alternatively and/or additionally, FIG. 6B depicts an example of the visual representation 650, which includes a visual indicator (e.g., a bounding box) configured to identify one or more corresponding portions 605 of the tumor sample within the image 300 and the distribution map 505.

In some example embodiments, the analysis engine 115 may also generate, for display in the user interface 135 at the client device 130, for example, a visual representation of one or more subpopulations of similar tiles present within the image 300 of the tumor sample. For example, each tile 305 in the image 300 may be associated with an x-quantity of pixels across one or more color channels (e.g., a single channel where the image 300 is a grayscale image, and three channels where the image 300 is a color image). Accordingly, in some cases, each tile 305 in the image 300 may be encoded as a vector of m values, each of which corresponding to an intensity value of a corresponding pixel in the image 300. In cases where the image 300 is a color image, the vector encoding each tile 305 may include, for each pixel in the image 300, a separate intensity value for each color channel (e.g., m=3x). One or more subpopulations of similar tiles in the image 300 of the tumor sample may be identified based on the pixel-wise representation of each tile 305 included in the image 300.

In some example embodiments, the analysis engine 115 may identify one or more subpopulations of similar tiles in the image 300 by applying, to a pixel-wise representation of each tile 305 in the image 300, a dimensionality reduction technique such as a principal component analysis (PCA), a uniform manifold approximation and projection (UMAP), a T-distributed Stochastic Neighbor Embedding (t-SNE), and/or the like. The resulting reduced dimension representation of the tiles 305 in the image 300 may correspond to a projection of the m-dimensional pixel-wise representation of each tile 305 onto a lower n-dimensional subspace (where n<<m).

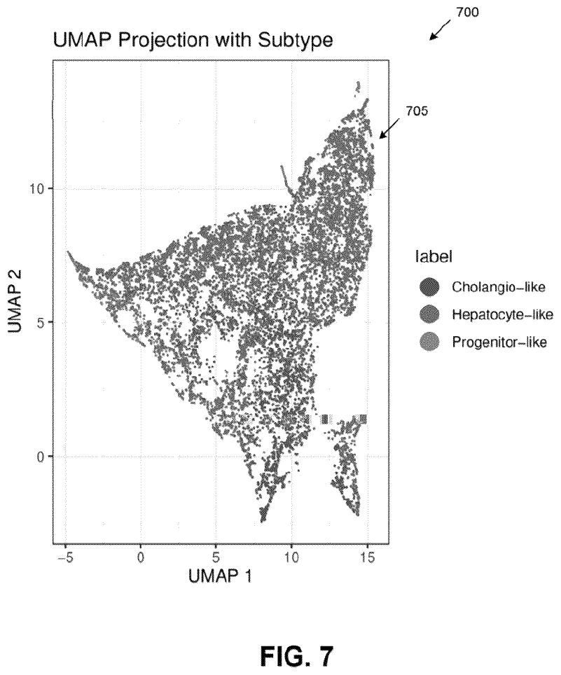

FIG. 7 depicts an example of a visual representation 700 that includes a reduced dimension representation 705 (e.g., a uniform manifold approximation and projection (UMAP)) of the tiles 305 included in the image 300. As shown in FIG. 7, the reduced dimension representation 705 may depict a distribution of the tiles 305 across a two-dimensional subspace. Moreover, FIG. 7 shows that each tile 305 in the reduced dimension representation 705 may be represented using a different visual indicator corresponding to the molecular subtype of the tile. Accordingly, symbols of different shapes, sizes, and/or colors may be used to enable a visual differentiation between, for example, tiles of the cholangio-like subtype, the progenitor-like subtype, and the hepatocyte-like subtype.

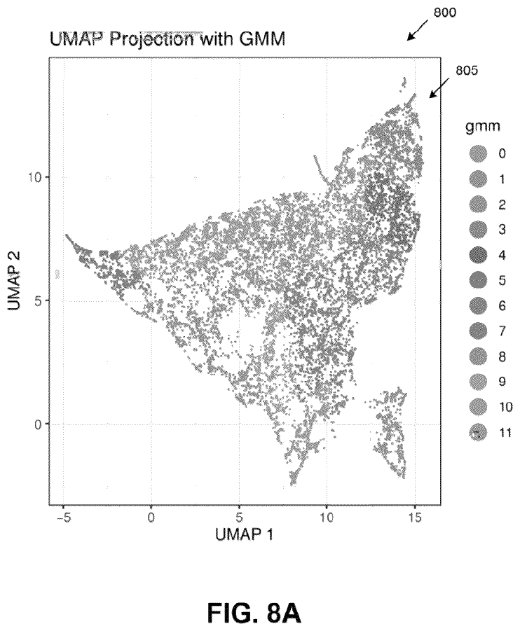

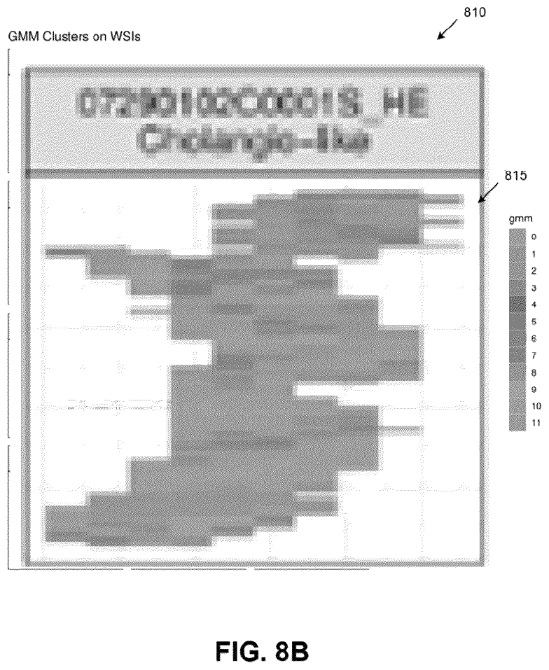

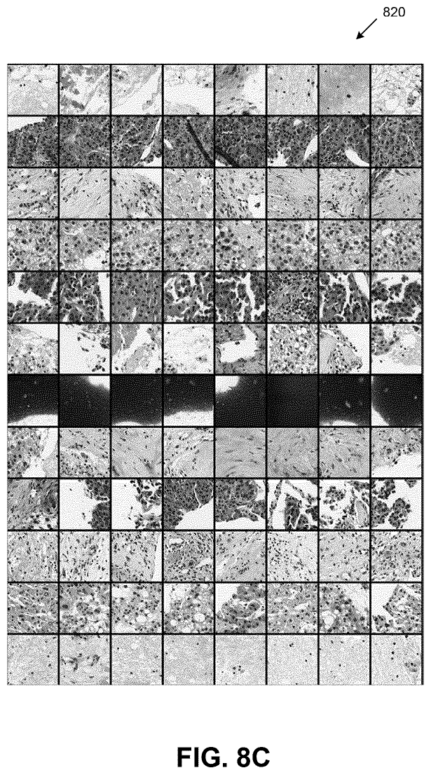

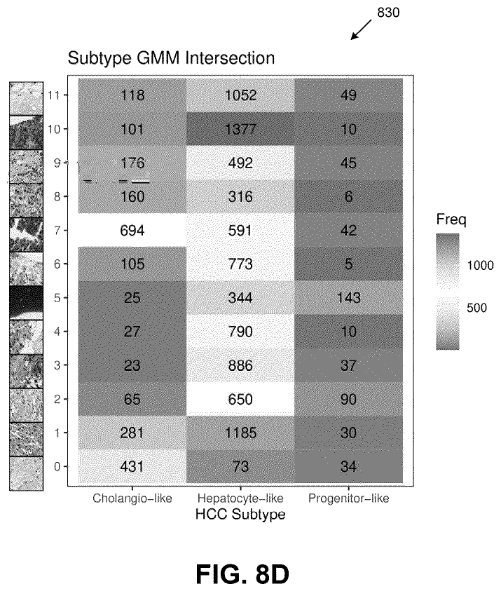

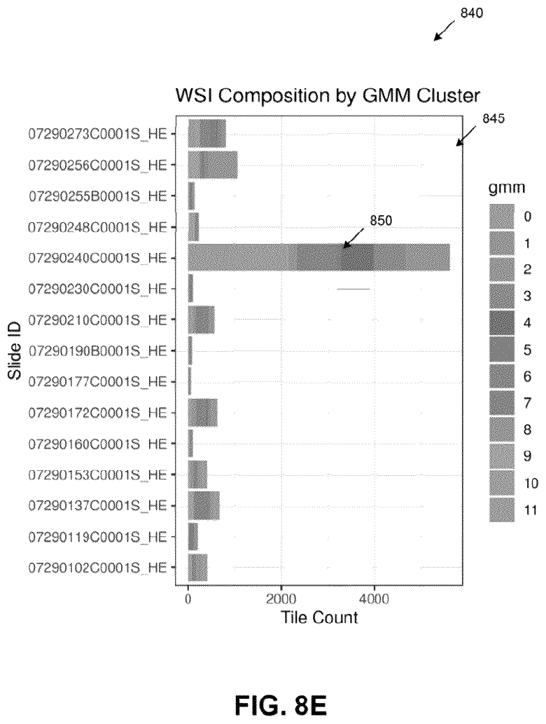

In some example embodiments, one or more subpopulations of similar tiles in the image 300 may be identified by applying, to the pixel-wise representation of each tile 305 in the image 300, a cluster analysis technique such as a k-means clustering, a mean-shift clustering, a density-based spatial clustering of applications with noise (DBSCAN), an expectation-maximization (EM) clustering using Gaussian mixture models (GMM), an agglomerative hierarchical clustering, and/or the like. In doing so, the analysis engine 115 may identify a quantity of clusters that maximizes the intra-cluster correlation amongst the members of each cluster. Moreover, the analysis engine 115 may identify a quantity of clusters associated with a minimum Bayesian information criteria, meaning that the distribution of the tiles 305 amongst the different clusters accurately reflects the distribution of the tiles 305. FIG. 8A depicts one example of a visual representation 800, which may be a distribution map 805 depicting the spatial distribution of the tiles assigned to different clusters of similar tiles along the lower n-dimensional subspace occupied by the reduced dimension representation of the tiles 305. The example of the visual representation 800 shown in FIG. 8A includes twelve different clusters (e.g., Gaussian mixture model (GMM) clusters), with members of each cluster being represented using different visual indicators (e.g., symbols of different colors, sizes, and/or shapes) in order to enable a visual differentiation therebetween. The twelve different clusters depicted in the visual representation 800 may be identified, for example, based on a lower-dimensional representation of the corresponding tiles. The mixture model (e.g., the Gaussian mixture model (GMM)) may represent the probability distribution of each grouping or subpopulation of similar tiles across the overall population as a whole.

FIG. 8B depicts another example of a visual representation 810, which may include a distribution map 815 showing the spatial distribution of tiles different clusters of similar tiles across the image 300 of the tumor sample. In the example shown in FIG. 8B, each tile 305 in the image 300 may be represented using a visual indicator corresponding to one of the twelve different clusters of similar tiles (e.g., Gaussian mixture model (GMM) clusters) associated with the tile. Accordingly, symbols of different shapes, sizes, and/or colors may be used to enable a visual differentiation between the tiles 305 may be represented using visual indicators that correspond to the cluster of the tiles from each cluster.

FIG. 8C depicts another example of a visual representation 820 in which the tiles 305 in the image 300 are arranged in accordance with their membership within the clusters of similar tiles. In the example of the visual representation 820 shown in FIG. 8C, each row of the grid may be occupied by tiles belonging to a separate cluster of similar tiles. As such, the visual representation 820 may provide a visual juxtaposition of similar tiles as well as dissimilar tiles within the image 300.

FIG. 8D depicts another example of a visual representation 830 depicting the distribution of different molecular subtypes across clusters of similar tiles, in accordance with some example embodiments. In the example shown in FIG. 8D, the visual representation 830 may provide textual as well as graphical indications of the frequency of each molecular subtype (e.g., the cholangio-like subtype, the progenitor-like subtype, and the hepatocyte-like subtype) within each cluster of similar tiles (e.g., Gaussian mixture model (GMM) clusters)). For example, in addition to the numerical values corresponding to the quantity of tiles that exhibit each molecular subtype within each cluster, each numerical value may be associated with a corresponding color to provide a heatmap display of the frequencies of each molecular subtype across the cluster of tiles.

FIG. 8E depicts another example of a visual representation 840 depicting a composition of various images of tumor samples in terms of the quantity of constituent tiles from each cluster of tiles, in accordance with some example embodiments. Referring to FIG. 8E, the visual representation 840 may include a bar graph 845 in which each bar 850 in the bar graph 845 corresponds to an image of a tumor sample such as, for example, the image 300. Moreover, as shown in FIG. 8E, each bar 850 in the bar graph 845 may include separate portions, each of which corresponding to a single cluster of similar tiles. Accordingly, each portion of the bar 850 in the bar graph 845 may have a length representative of the quantity of tiles that belong to a corresponding cluster of similar tiles.

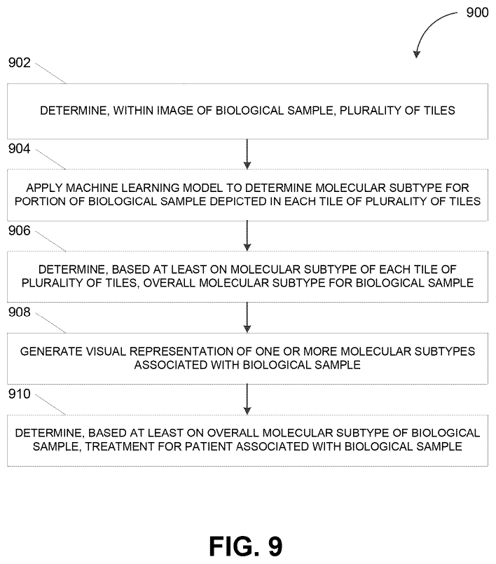

FIG. 9 depicts a flowchart illustrating an example of a process 900 for image-based molecular subtype classification, in accordance with some example embodiments. For instance, in some example embodiments, the analysis engine 115 at the digital pathology platform 110 may perform the process 900 to determine, based at least on an image of a tumor sample received from the imaging system 120, one or more molecular subtypes present in the tumor sample. In some cases, the analysis engine 115 may further perform the process 900 to determine, based at least on the molecular subtypes present in the tumor sample, a treatment for a patient associated with the tumor sample.

At 902, the analysis engine 115 may determine, within an image of a biological sample, a plurality of tiles. For instance, as shown in FIGS. 3A-B, the analysis engine 115 may determine, within the image 300 of the tumor sample, one or more tiles 305 including, for example, a first tile 305a, a second tile 305b, and/or the like. The image 300 may depict the cells of the tumor sample, in which case each tile 305 may depict at least a portion of the cells included in the tumor sample. Moreover, in some cases, the analysis engine 115 may exclude, from further image-based molecular subtype classification, tiles that do not depicts an above threshold quantity of the cells present in the tumor sample. For instance, tiles with an above-threshold proportion of a background of the image and/or a below-threshold mean color channel variance (e.g., gray colored tiles) may be excluded from further image-based molecular subtype classification.

At 904, the analysis engine 115 may apply a machine learning model to determine a molecular subtype for a portion of the biological sample depicted in each tile of the plurality of tiles. In some example embodiments, the analysis engine 115 may apply a machine learning model, such as the machine learning model 400 shown in FIG. 4, to determine a molecular subtype for each tile 305 in the image 300. As noted, in some cases, the machine learning model 400 may be an artificial neural network (or another type of machine learning model) trained to recognize the morphological patterns that are associated with each molecular subtype.

At 906, the analysis engine 115 may determine, based at least on the molecular subtype of each tile of the plurality of tiles, an overall molecular subtype for the biological sample. In some example embodiments, the analysis engine 115 may determine the overall molecular subtype of the tumor sample depicted in the image 300 based at least on the quantity of each molecular subtype present within the tumor sample. Alternatively and/or additionally, the analysis engine 115 may apply another machine learning model to determine, based at least on the molecular subtype of each tile 305, the overall molecular subtype of the tumor sample depicted in the image 300. This other machine learning model may be a multiple instance learning (MIL) model trained to determine the overall molecular subtype by determining a representational encoding of the tiles 305 included in the image 300.

In some cases, this other machine learning model may include an attention mechanism configured to assign, to each tile 305, an attention score representative of how relevant the molecular subtype of each tile 305 is to the overall molecular subtype of the image 300 of the tumor sample. Accordingly, the first tile 305a may be assigned a higher attention score than the second tile 305b if the first molecular subtype of the first tile 305 is more relevant to the overall molecular subtype of the image 300 than the second molecular subtype of the second tile 305b.

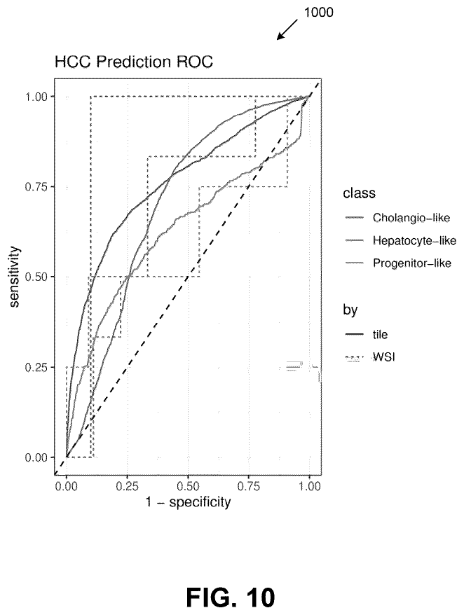

It should be appreciated that the aforementioned machine learning enabled technique for image-based molecular subtype classification may improve the accuracy of image-based molecular subtype classification by at least minimizing outcome variability associated with conventional digital pathology approaches. To further illustrate, FIG. 10 depicts a graph 1000 illustrating an receiver operating characteristic (ROC) curve representative of the performance of the machine learning enabled image-based molecular subtype classification technique described herein when applied to hepatocellular carcinoma (HCC) tumor samples. As shown in the graph 1000, the machine learning based approach achieved an area under the curve (AUC) of 0.698 for individual tile molecular subtype classification and an area under the curve (AUC) of 0.733 for overall image molecular subtype classification. These high area under the curve (AUC) values indicate that the machine learning based approach described herein is able to differentiate between different hepatocellular carcinoma (HCC) molecular subtypes with high precision.

At 908, the analysis engine 115 may generate one or more visual representation of one or more molecular subtypes associated with the biological sample. In some example embodiments, the analysis engine 115 may generate a visual representation that depicts a spatial distribution of different molecular subtypes within the biological sample depicted in the image 300 (e.g., FIG. 5B). Alternatively and/or additionally, the analysis engine 115 may generate a visual representation in which the tiles 305 of the image 300 are organized in accordance with their corresponding molecular subtypes (e.g., FIG. 5A). In some cases, the analysis engine 115 may generate a visual representation that depicts various relationships between subpopulations of similar tiles within the image 300 (e.g., FIGS. 7 and 8A-E). For instance, the analysis engine 115 may identify one or more subpopulations of similar tiles by applying a dimensionality reduction technique, a cluster analysis technique, and/or the like. Furthermore, the analysis engine 115 may generate one or more corresponding visual representations that depict the distribution of molecular subtypes across the subpopulations of similar tiles, the distribution of similar tiles across the tumor sample in the image 300, and/or the like.

At 910, the analysis engine 115 may determine, based at least on the overall molecular subtype for the biological sample, a treatment for a patient associated with the biological sample. As noted, the molecular subtypes of certain cancers, including hepatocellular carcinoma (HCC) may serve as a crucial biomarker for predicting patient response to therapy and survival. Accordingly, in cases where the tumor sample is a hepatocellular carcinoma (HCC) tumor sample, for example, the analysis engine 115 may determine whether the hepatocellular carcinoma (HCC) tumor sample is associated with a cholangio-like subtype, a progenitor-like subtype, or a hepatocyte-like subtype. Moreover, the molecular subtype of the hepatocellular carcinoma tumor sample may be used to determine whether the treatment for the patient associated with the hepatocellular tumor sample should include combination immunotherapy, such as an atezolizumab (anti-PD-L1) plus bevacizumab (anti-VEGF) combination therapy, and additional therapies to overcome subtype-specific resistances to certain therapies (e.g., an GPC3/CD3 bi-specific antibody to overcome the resistance to combination immunotherapy associated with the progenitor-like subtype).

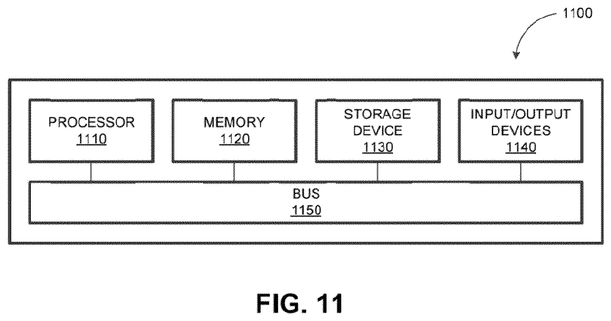

FIG. 11 depicts a block diagram illustrating an example of computing system 1100, in accordance with some example embodiments. Referring to FIGS. 1 and 11, the computing system 1100 may be used to implement the digital pathology platform 110, the client device 130, and/or any components therein.

As shown in FIG. 11, the computing system 1100 can include a processor 1110, a memory 1120, a storage device 1130, and input/output device 1140. The processor 1110, the memory 1120, the storage device 1130, and the input/output device 1140 can be interconnected via a system bus 1150. The processor 1110 is capable of processing instructions for execution within the computing system 1100. Such executed instructions can implement one or more components of, for example, the digital pathology platform 110, the client device 130, and/or the like. In some example embodiments, the processor 1110 can be a single-threaded processor. Alternately, the processor 1110 can be a multi-threaded processor. The processor 1110 is capable of processing instructions stored in the memory 1120 and/or on the storage device 1130 to display graphical information for a user interface provided via the input/output device 1140.

The memory 1120 is a computer readable medium such as volatile or non-volatile that stores information within the computing system 1100. The memory 1120 can store data structures representing configuration object databases, for example. The storage device 1130 is capable of providing persistent storage for the computing system 1100. The storage device 1130 can be a floppy disk device, a hard disk device, an optical disk device, or a tape device, or other suitable persistent storage means. The input/output device 1140 provides input/output operations for the computing system 1100. In some example embodiments, the input/output device 1140 includes a keyboard and/or pointing device. In various implementations, the input/output device 1140 includes a display unit for displaying graphical user interfaces.

According to some example embodiments, the input/output device 1140 can provide input/output operations for a network device. For example, the input/output device 1140 can include Ethernet ports or other networking ports to communicate with one or more wired and/or wireless networks (e.g., a local area network (LAN), a wide area network (WAN), the Internet).

In some example embodiments, the computing system 1100 can be used to execute various interactive computer software applications that can be used for organization, analysis and/or storage of data in various formats. Alternatively, the computing system 1100 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1140. The user interface can be generated and presented to a user by the computing system 1100 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

### Embodiments

Among the provided embodiments are:

1. A system, comprising:
at least one data processor; and
at least one memory storing instructions, which when executed by the at least one data processor, result in operations comprising:
   determining, within an image of a biological sample, a plurality of tiles, each tile of the plurality of tiles depicting a portion of the biological sample;
   applying a first machine learning model to determine a molecular subtype for the portion of the biological sample depicted in each tile of the plurality of tiles; and
   determining, based at least on the molecular subtype of each tile of the plurality of tiles, an overall molecular subtype for the biological sample.

2. The system of embodiment 1, wherein the overall molecular subtype of the biological sample is determined by applying a second machine learning model.

3. The system of embodiment 2, wherein the second machine learning model is trained to determine the overall molecular subtype by at least determining a representational encoding of the plurality of tiles.

4. The system of embodiment 3, wherein the second machine learning model is further trained to assign, to a first tile of the plurality of tiles, a higher attention score than a second tile of the plurality of tiles while determining the representational encoding of the plurality of tiles.

5. The system of embodiment 4, wherein the higher attention score indicates that a first molecular subtype of the first tile contributes more to the representational encoding of the plurality of tiles than a second molecular subtype of the second tile.

6 The system of any one of embodiments 4 to 5, wherein the higher attention score indicates that a first molecular subtype of the first tile is more relevant to the overall molecular subtype of the biological sample than a second molecular subtype of the second tile.

7. The system of any one of embodiments 2 to 6, wherein the second machine learning model comprises a multiple instance learning (MIL) model.

8. The system of any one of embodiments 2 to 7, wherein the second machine learning model includes an attention mechanism.

9. The system of any one of embodiments 1 to 8, wherein the operations further comprise:
generating a first visual representation of a reduced dimension representation of the plurality of tiles.

10. The system of embodiment 9, wherein the first visual representation includes one or more visual indicators configured to provide a visual differentiation between tiles of different subtypes.

11. The system of any one of embodiments 9 to 10, wherein the first visual representation is generated by at least applying, to a pixel-wise representation of each tile of the plurality of tiles, a dimensionality reduction technique.

12. The system of embodiment 11, wherein the dimensionality reduction technique includes one or more of a principal component analysis (PCA), a uniform manifold approximation and projection (UMAP), and a T-distributed Stochastic Neighbor Embedding (t-SNE).

13. The system of any one of embodiments 9 to 12, wherein the first visual representation is further generated to include one or more visual indications configured to provide a visual differentiation between one or more clusters of similar tiles within the plurality of tiles.

14. The system of embodiment 13, wherein the operations further comprise:
generating a second visual representation depicting the plurality of tiles organized in accordance with the one or more clusters of similar tiles.

15. The system of any one of embodiments 13 to 14, wherein the operations further comprise:
generating a second visual representation depicting a spatial distribution of the one or more clusters of similar tiles within the biological sample.

16. The system of any one of embodiments 13 to 15, wherein the one or more clusters of similar tiles are identified by applying a cluster analysis technique.

17. The system of embodiment 16, wherein the cluster analysis technique includes one or more of a k-means clustering, a mean-shift clustering, a density-based spatial clustering of applications with noise (DBSCAN), an expectation-maximization (EM) clustering using Gaussian mixture models (GMM), and an agglomerative hierarchical clustering.

18. The system of any one of embodiments 1 to 17, wherein the overall molecular subtype of the biological sample is determined based at least on a quantity of each molecular subtype present within the plurality of cells.

19. The system of any one of embodiments 1 to 18, wherein the operations further comprise:
generating, based at least on the molecular subtype of each tile of the plurality of tiles, a visual representation depicting a spatial distribution of one or more molecular subtypes within the biological sample.

20. The system of any one of embodiments 1 to 19, wherein the operations further comprise:
generating a visual representation depicting a first tile of the plurality of tiles having a first subtype along with a second tile of the first subtype from a same biological sample or a different biological sample.

21. The system of embodiment 20, wherein the visual representation is further generated to depict a third tile of the plurality of tiles having a second subtype along with a fourth tile of the second subtype from the same biological sample or the different biological sample.

22. The system of any one of embodiments 1 to 21, wherein the plurality of tiles exclude one or more tiles in the image with an above-threshold proportion of a background of the image or a below-threshold mean color channel variance.

23. The system of any one of embodiments 1 to 22, wherein the first machine learning model is trained to determine the molecular subtype associated with each tile of the plurality of tiles based on a morphological pattern present within the portion of the biological sample depicted in each tile.

24. The system of any one of embodiments 1 to 23, wherein the first machine learning model comprises an artificial neural network (ANN).

25. The system of any one of embodiments 1 to 24, wherein the biological sample comprises a hepatocellular carcinoma (HCC) tissue sample, wherein each tile of the plurality of tiles is assigned a molecular subtype comprising one of a cholangio-like subtype, a hepatocyte-like subtype, or a progenitor-like subtype, and wherein the overall molecular subtype of the plurality of cells depicted in the image of the biological sample comprises one of the cholangio-like subtype, the hepatocyte-like subtype, or the progenitor-like subtype.

26. The system of any one of embodiments 1 to 25, wherein the operations further comprise:
identifying, based at least on transcriptome data associated with a plurality of tumor tissue samples, a plurality of molecular subtypes.

27. The system of embodiment 26, wherein the plurality of tumor tissue samples comprises a plurality of hepatocellular carcinoma (HCC) tumor tissue samples, and wherein the plurality of molecular subtypes includes a cholangio-like subtype, a hepatocyte-like subtype, and a progenitor-like subtype.

28. The system of any one of embodiments 26 to 27, wherein the first machine learning model is trained to assign, to each tile of the plurality of tiles, a label corresponding to one of the plurality of molecular subtypes identified based on the transcriptome data.

29. The system of any one of embodiments 26 to 28, wherein the overall molecular subtype of the plurality of cells depicted in the image of the biological sample comprises one of the plurality of molecular subtypes identified based on the transcriptome data.

30. The system of any one of embodiments 1 to 29, wherein the image depicts a plurality of cells comprising the biological sample, and wherein each tile of the plurality of tiles depict a portion of the plurality of cells comprising the biological sample.

31. A computer-implemented method, comprising:
determining, within an image of a biological sample, a plurality of tiles, each tile of the plurality of tiles depicting a portion of the biological sample;
applying a first machine learning model to determine a molecular subtype for the portion of the biological sample depicted in each tile of the plurality of tiles; and
determining, based at least on the molecular subtype of each tile of the plurality of tiles, an overall molecular subtype for the biological sample.

32. The method of embodiment 31, wherein the overall molecular subtype of the biological sample is determined by applying a second machine learning model.

33. The method of embodiment 32, wherein the second machine learning model is trained to determine the overall molecular subtype by at least determining a representational encoding of the plurality of tiles.

34. The method of embodiment 33, wherein the second machine learning model is further trained to assign, to a first tile of the plurality of tiles, a higher attention score than a second tile of the plurality of tiles while determining the representational encoding of the plurality of tiles.

35. The method of embodiment 34, wherein the higher attention score indicates that a first molecular subtype of the first tile contributes more to the representational encoding of the plurality of tiles than a second molecular subtype of the second tile.

36. The method of any one of embodiments 34 to 35, wherein the higher attention score indicates that a first molecular subtype of the first tile is more relevant to the overall molecular subtype of the biological sample than a second molecular subtype of the second tile.

37. The method of any one of embodiments 32 to 36, wherein the second machine learning model comprises a multiple instance learning (MIL) model.

38. The method of any one of embodiments 32 to 37, wherein the second machine learning model includes an attention mechanism.

39. The method of any one of embodiments 31 to 38, further comprising:
generating a first visual representation of a reduced dimension representation of the plurality of tiles.

40. The method of embodiment 39, wherein the first visual representation includes one or more visual indicators configured to provide a visual differentiation between tiles of different subtypes.

41. The method of any one of embodiments 39 to 40, wherein the first visual representation is generated by at least applying, to a pixel-wise representation of each tile of the plurality of tiles, a dimensionality reduction technique.

42. The method of embodiment 41, wherein the dimensionality reduction technique includes one or more of a principal component analysis (PCA), a uniform manifold approximation and projection (UMAP), and a T-distributed Stochastic Neighbor Embedding (t-SNE).

43. The method of any one of embodiments 39 to 42, wherein the first visual representation is further generated to include one or more visual indications configured to provide a visual differentiation between one or more clusters of similar tiles within the plurality of tiles.

44. The method of embodiment 43, further comprising:
generating a second visual representation depicting the plurality of tiles organized in accordance with the one or more clusters of similar tiles.

45. The method of any one of embodiments 43 to 44, further comprising:
generating a second visual representation depicting a spatial distribution of the one or more clusters of similar tiles within the biological sample.

46. The method of any one of embodiments 43 to 45, wherein the one or more clusters of similar tiles are identified by applying a cluster analysis technique.

47. The method of embodiment 46, wherein the cluster analysis technique includes one or more of a k-means clustering, a mean-shift clustering, a density-based spatial clustering of applications with noise (DBSCAN), an expectation-maximization (EM) clustering using Gaussian mixture models (GMM), and an agglomerative hierarchical clustering.

48. The method of any one of embodiments 31 to 47, wherein the overall molecular subtype of the biological sample is determined based at least on a quantity of each molecular subtype present within the plurality of cells.

49. The method of any one of embodiments 31 to 48, further comprising:
generating, based at least on the molecular subtype of each tile of the plurality of tiles, a visual representation depicting a spatial distribution of one or more molecular subtypes within the biological sample.

50. The method of any one of embodiments 31 to 49, further comprising:
generating a visual representation depicting a first tile of the plurality of tiles having a first subtype along with a second tile of the first subtype from a same biological sample or a different biological sample.

51. The method of embodiment 50, wherein the visual representation is further generated to depict a third tile of the plurality of tiles having a second subtype along with a fourth tile of the second subtype from the same biological sample or the different biological sample.

52. The method of any one of embodiments 31 to 51, wherein the plurality of tiles exclude one or more tiles in the image with an above-threshold proportion of a background of the image or a below-threshold mean color channel variance.

53. The method of any one of embodiments 31 to 52, wherein the first machine learning model is trained to determine the molecular subtype associated with each tile of the plurality of tiles based on a morphological pattern present within the portion of the biological sample depicted in each tile.

54. The method of any one of embodiments 31 to 53, wherein the first machine learning model comprises an artificial neural network (ANN).

55. The method of any one of embodiments 31 to 54, wherein the biological sample comprises a hepatocellular carcinoma (HCC) tissue sample, wherein each tile of the plurality of tiles is assigned a molecular subtype comprising one of a cholangio-like subtype, a hepatocyte-like subtype, or a progenitor-like subtype, and wherein the overall molecular subtype of the plurality of cells depicted in the image of the biological sample comprises one of the cholangio-like subtype, the hepatocyte-like subtype, or the progenitor-like subtype.

56. The method of any one of embodiments 31 to 55, further comprising:
identifying, based at least on transcriptome data associated with a plurality of tumor tissue samples, a plurality of molecular subtypes.

57. The method of embodiment 56, wherein the plurality of tumor tissue samples comprises a plurality of hepatocellular carcinoma (HCC) tumor tissue samples, and wherein the plurality of molecular subtypes includes a cholangio-like subtype, a hepatocyte-like subtype, and a progenitor-like subtype.

58. The method of any one of embodiments 56 to 57, wherein the first machine learning model is trained to assign, to each tile of the plurality of tiles, a label corresponding to one of the plurality of molecular subtypes identified based on the transcriptome data.

59. The method of any one of embodiments 56 to 58, wherein the overall molecular subtype of the plurality of cells depicted in the image of the biological sample comprises one of the plurality of molecular subtypes identified based on the transcriptome data.

60. The method of any one of embodiments 31 to 59, wherein the image depicts a plurality of cells comprising the biological sample, and wherein each tile of the plurality of tiles depict a portion of the plurality of cells comprising the biological sample.

61. A non-transitory computer readable medium storing instructions, which when executed by at least one data processor, result in operations comprising:
determining, within an image of a biological sample, a plurality of tiles, each tile of the plurality of tiles depicting a portion of the biological sample;
applying a first machine learning model to determine a molecular subtype for the portion of the biological sample depicted in each tile of the plurality of tiles; and determining, based at least on the molecular subtype of each tile of the plurality of tiles, an overall molecular subtype for the biological sample.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B:” “one or more of A and B:” and “A and/or B” are each intended to mean “A alone, B alone, or A and B together.” A similar interpretation is also intended for lists including three or more items. For example, the phrases “at least one of A, B, and C:” “one or more of A, B, and C:” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A system, comprising:
at least one data processor; and
at least one memory storing instructions, which when executed by the at least one data processor, result in operations comprising:
determining, within an image of a biological sample, a plurality of tiles, each tile of the plurality of tiles depicting a portion of the biological sample;
applying a first machine learning model to determine a molecular subtype for the portion of the biological sample depicted in each tile of the plurality of tiles; and
determining, based at least on the molecular subtype of each tile of the plurality of tiles, an overall molecular subtype for the biological sample.

2. The system of claim 1, wherein the overall molecular subtype of the biological sample is determined by applying a second machine learning model.

3. The system of claim 2, wherein the second machine learning model is trained to determine the overall molecular subtype by at least determining a representational encoding of the plurality of tiles.

4. The system of claim 2, wherein the second machine learning model comprises a multiple instance learning (MIL) model.

5. The system of claim 1, wherein the operations further comprise:
generating a first visual representation of a reduced dimension representation of the plurality of tiles.

6. The system of claim 1, wherein the overall molecular subtype of the biological sample is determined based at least on a quantity of each molecular subtype present within the plurality of cells.

7. The system of claim 1, wherein the operations further comprise:
generating, based at least on the molecular subtype of each tile of the plurality of tiles, a visual representation depicting a spatial distribution of one or more molecular subtypes within the biological sample.

8. The system of claim 1, wherein the operations further comprise:
generating a visual representation depicting a first tile of the plurality of tiles having a first subtype along with a second tile of the first subtype from a same biological sample or a different biological sample.

9. The system of claim 1, wherein the plurality of tiles exclude one or more tiles in the image with an above-threshold proportion of a background of the image or a below-threshold mean color channel variance.

10. The system of claim 1, wherein the first machine learning model is trained to determine the molecular subtype associated with each tile of the plurality of tiles based on a morphological pattern present within the portion of the biological sample depicted in each tile.

11. The system of claim 1, wherein the first machine learning model comprises an artificial neural network (ANN).

12. The system of claim 1, wherein the biological sample comprises a hepatocellular carcinoma (HCC) tissue sample, wherein each tile of the plurality of tiles is assigned a molecular subtype comprising one of a cholangio-like subtype, a hepatocyte-like subtype, or a progenitor-like subtype, and wherein the overall molecular subtype of the plurality of cells depicted in the image of the biological sample comprises one of the cholangio-like subtype, the hepatocyte-like subtype, or the progenitor-like subtype.

13. The system of claim 1, wherein the operations further comprise:
identifying, based at least on transcriptome data associated with a plurality of tumor tissue samples, a plurality of molecular subtypes.

14. The system of claim 1, wherein the image depicts a plurality of cells comprising the biological sample, and wherein each tile of the plurality of tiles depict a portion of the plurality of cells comprising the biological sample.

15. A computer-implemented method, comprising:
determining, within an image of a biological sample, a plurality of tiles, each tile of the plurality of tiles depicting a portion of the biological sample;
applying a first machine learning model to determine a molecular subtype for the portion of the biological sample depicted in each tile of the plurality of tiles; and
determining, based at least on the molecular subtype of each tile of the plurality of tiles, an overall molecular subtype for the biological sample.

16. The method of claim 15, wherein the overall molecular subtype of the biological sample is determined by applying a second machine learning model.

17. The method of claim 16, wherein the second machine learning model is trained to determine the overall molecular subtype by at least determining a representational encoding of the plurality of tiles.

18. The method of claim 16, wherein the second machine learning model comprises a multiple instance learning (MIL) model.

19. The method of claim 15, further comprising:
generating a first visual representation of a reduced dimension representation of the plurality of tiles.

20. The method of claim 15, wherein the overall molecular subtype of the biological sample is determined based at least on a quantity of each molecular subtype present within the plurality of cells.

21. The method of claim 15, further comprising:
generating, based at least on the molecular subtype of each tile of the plurality of tiles, a visual representation depicting a spatial distribution of one or more molecular subtypes within the biological sample.

22. The method of claim 15, further comprising:
generating a visual representation depicting a first tile of the plurality of tiles having a first subtype along with a second tile of the first subtype from a same biological sample or a different biological sample.

23. The method of claim 15, wherein the plurality of tiles exclude one or more tiles in the image with an above-threshold proportion of a background of the image or a below-threshold mean color channel variance.

24. The method of claim 15, wherein the first machine learning model is trained to determine the molecular subtype associated with each tile of the plurality of tiles based on a morphological pattern present within the portion of the biological sample depicted in each tile.

25. The method of claim 15, wherein the first machine learning model comprises an artificial neural network (ANN).

26. The method of claim 15, wherein the biological sample comprises a hepatocellular carcinoma (HCC) tissue sample, wherein each tile of the plurality of tiles is assigned a molecular subtype comprising one of a cholangio-like subtype, a hepatocyte-like subtype, or a progenitor-like subtype, and wherein the overall molecular subtype of the plurality of cells depicted in the image of the biological sample comprises one of the cholangio-like subtype, the hepatocyte-like subtype, or the progenitor-like subtype.

27. The method of claim 15, further comprising:
identifying, based at least on transcriptome data associated with a plurality of tumor tissue samples, a plurality of molecular subtypes.

28. The method of claim 15, wherein the image depicts a plurality of cells comprising the biological sample, and wherein each tile of the plurality of tiles depict a portion of the plurality of cells comprising the biological sample.
